(19) United States
(12) Patent Application Publication
(10) Pub. No.: US 2009/0062875 A1
(43) Pub. Date: Mar. 5, 2009

(54) Miniature defibrillator

(76) Inventors: Daniel Gelbart, Vancouver (CA); Samuel Victor Lichtenstein, Vancouver (CA)

Correspondence Address: DANIEL GELBART, 4706 DRUMMOND DR, VANCOUVER, BC, V6T-1B4, CA

(21) Appl. No.: 12/215,163

(22) Filed: Jun. 26, 2008

Related U.S. Application Data
(63) Continuation-in-part of application No. 11/896,172, filed on Aug. 30, 2007, now abandoned.

(51) Int. Cl. A61N 1/39

(52) U.S. Cl. 607/7

(57) ABSTRACT

A miniature implanted defibrillator ignites an explosive charge when it senses an erratic heart rhythm. The defibrillator can be delivered percutaneously into the heart or can be implanted in the vicinity of the heart via minimally invasive surgery. The shock created by the exploding charge defibrillates the heart. Single use and multiple use devices are possible. The same principle can be used for a disposable external defibrillators.

### FIELD OF THE INVENTION

The invention is in the medical field and more specifically is related to implantable cardiac defibrillators

### BACKGROUND OF THE INVENTION

Defibrillators are used to re-start the heart in case of erratic rhythm (fibrillation) or cessation of pacing. There are two types of defibrillators: external and internal (implanted). Since the energy required to shock the heart and re-start it is in the range of tens of Joules, the implanted defibrillators rely on a large energy storage capacitor. A battery is keeping this capacitor charges but the leakage current causes a battery drain. The size of capacitor and battery requires implantation by surgery. It is desirable to have a defibrillator sufficiently small to be delivered percutaneously via a catheter. Since defibrillators can be viewed as a preventive measure, and many of the installed ones are never activated, more would be used if the installation was simpler. It is also desirable to have a device with very low battery drain so it can have a long lifetime. The high energy density and reliability of explosive devices have been known for many years, and indeed some external defibrillators designs use explosives to drive a generator in order to overcome the battery lifetime limit. The energy density of an explosive charge is about 100 to 1000 times higher than that of a capacitor, allowing great reduction in size if a defibrillator could be driven directly by the explosive charge, without the need to convert it to electricity. It has been known in medicine that a sharp blow to the chest, known as “precordial thump” can act as a defibrillator. This effect is used by the current invention.

### SUMMARY OF THE DISCLOSURE

A miniature implanted defibrillator ignites an explosive charge when it senses an erratic heart rhythm. The defibrillator can be delivered percutaneously into the heart or can be implanted in the vicinity of the heart via minimally invasive surgery. The shock created by the exploding charge defibrillates the heart. Single use and multiple use devices are possible. The same principle can be used for disposable external defibrillators.

### DESCRIPTION OF THE DRAWINGS

FIG. 1 is a schematic cross section of the heart showing the defibrillator being deployed via a catheter into the right ventricle.

FIG. 2 is a longitudinal cross section of the defibrillator.

FIG. 3 is a front view of the defibrillator.

FIG. 4 is an isometric view of a defibrillator capable of multiple activations.

FIG. 5 is an electrical schematic diagram of the defibrillator.

FIG. 6 is a cross section of an external defibrillator according to the disclosure.

### DETAILED DESCRIPTION

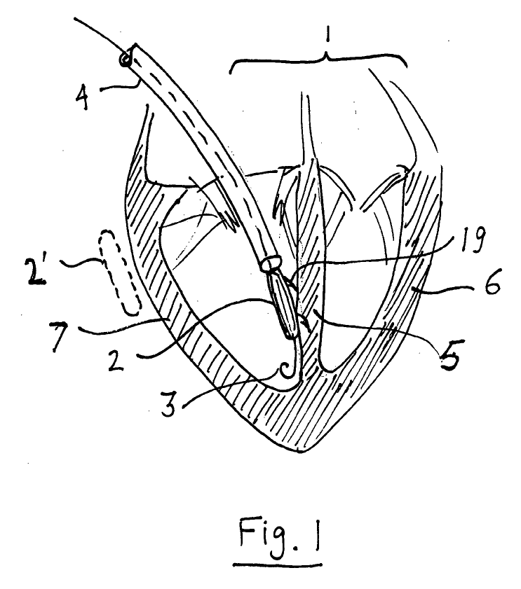

The amount of energy required to defibrillate a heart is in the range of 1 J to 200 J, depending on the exact point the energy is applied. Most defibrillators used today apply a pulse of 10 J-100 J, generated from discharging a capacitor. The volume of such a capacitor is many cubic centimeters. The same amount of energy can be supplied by an explosive charge of 10-100 mg and having a negligible volume. This allows building a defibrillator sufficiently small to be delivered percutaneously via a catheter directly into the heart. Since the principle of operation is mechanical, not electrical, no leads are required. Eliminating the leads further simplifies the process and reduces infection as well as other risks. FIG. 1 shows a miniature defibrillator 2 delivered into a heart 1 via catheter 4. A guide wire 3 can be used. The art of percutaneous delivery of cardiac implants is well known. The defibrillator 2 can be attached to the septum 6, left ventricular wall 6 or right ventricular wall 7. It is attached to the inside of the heart by embedding barbs 19 into the tissue. It can also be located outside the heart, but in proximity to it, as shown by 2′. In this case a minimally invasive procedure is needed. The incision can be small as the diameter of the preferred embodiment of the defibrillator is in the range of 5-8 mm.

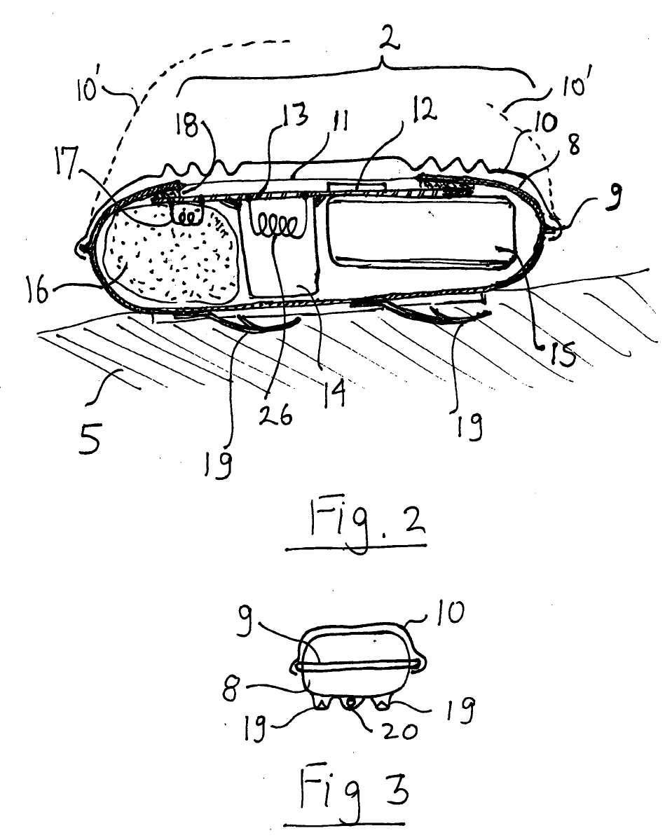

FIG. 2 shows a cross section of the defibrillator 2. A metal case 8 is hermetically sealed by weld 9. One section 11 of the case is made very thin in order to allow sensing of the heartbeats and allow the explosion to easily burst this section. It is desirable to cover this section with a flexible and somewhat porous cover 10 for two reasons: to contain any contamination created by the explosion and to contain the gas bubble created by the explosion. The gas bubble will disappear slowly by being dissolved in the blood stream. A single large bubble released into the blood stream, even on the venous side, could be dangerous. The electronic circuit is built on substrate 13 (preferable as a monolithic custom made silicon IC). It may contain, among others, capacitive sensing electrode 12, battery 15, storage capacitor 14, explosive charge 16, igniter 17, adhesive bond 18 and inductive coupling coil 26. Once the defibrillator is activated, thin section 11 ruptures and the generated gas inflates porous cover 10 to position 10′. The typical gas volume created is 2-20 cc. Any gas permeable elastomeric material having long-term blood compatibility can be used for cover 10, as long as it can act as an inflatable balloon. By the way of example, a thin polyurethane balloon can be used. Folds or convolutions in this cover aid expansion for less elastomeric materials.

The defibrillator is attached to the tissue via flexible barbs 19 by pushing it forward and embedding the barbs into the cardiac tissue, as shown in FIG. 1. The operation of the defibrillator is not affected by being covered up by tissue. The exterior may be treated to promote tissue growth in order to minimize the need for anti-clotting drugs. Other coatings can be drug eluting coating, anti-inflammatory surfaces or any other beneficial combination of surface finishes and coatings.

The variations in blood pressure during the cardiac cycle cause wall 11 to flex slightly, changing the capacitance between wall 11 and sensing electrode 12. This change can be used to detect the heartbeats. Other detection methods, such as strain gages, magnetic, piezoelectric, accelerometer, optical etc can also be used, as long as the required power is very low, preferably under 1 uW. In the alternative, barbs 19 can be electrodes sensing the cardiac rhythm by picking up the electrical activity from the cardiac wall.

FIG. 3 shows a front view of the defibrillator. Hole 20 is used to slide the device over a guide wire.

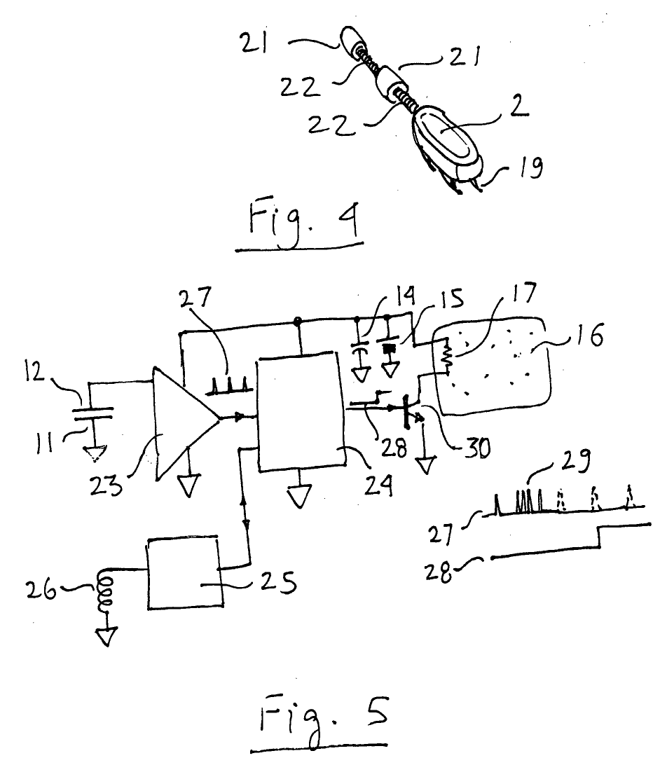

In its simplest form the device is a “single use” device, i.e. can handle only a single event. By using several explosive cartridges connected to a single electronic module, multiple events can be handled. This is shown in FIG. 4. The additional cartridges 21 are connected to defibrillator 2 via hermetically sealed wired 22. To achieve a reliable seal the preferred method is to enclose wires in a miniature rigid or flexible metal tube 22 such as a miniature bellows. This is needed to maintain hermetic seal. Inside the bellows the wires have to pass hermetic seals, such as glass to metal seals, as well. The reason for separating the cartridges 21 is to avoid damage to the unexploded ones. Obviously the one farthest from the main module 2 is used first.

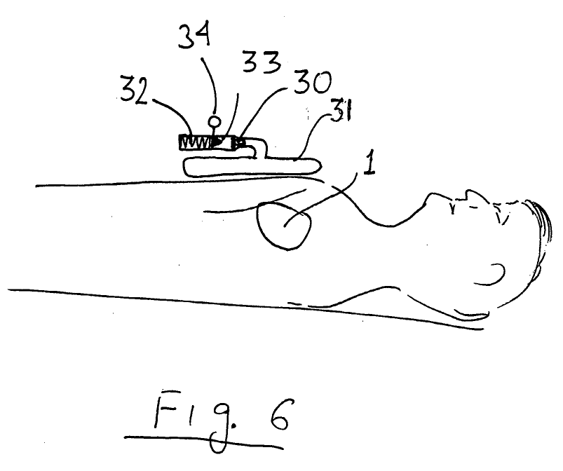

FIG. 5 details the electronic circuit. Heartbeat sensor, such as capacitive sensor 12 sensing motion of thin wall 11, is connected to amplifier 23 generating pulses 17 corresponding to the heartbeats. Logic circuit 24 is programmed to activate switch 30 and ignite charge 16 by heating up igniter wire 17 (typically thin Nichrome wire) when detecting an abnormal activity in pulses 17 such as fibrillation or lack of pulses.

Capacitor 14 is used to allow a momentary high current into igniter 17 even when battery 14 is old and has high internal resistance. Battery 14 is preferably of the Lithium-Ion type. Logic circuit 24 can be programmed to wait a set amount of time before activating defibrillator when detecting an erratic cardiac rhythm. This is shown by timing diagram 29 showing input waveform 27 to logic circuit 24 and output 28. Typical activation delays are in the range of 1-10 seconds.

Arming of the device after delivery, as well as changing of programmed parameters, can be done by inductive coupling. A small coil 26 is used as a receiver. Signal from coil is rectified and converted to logic levels by receiver 25 and fed to logic circuit 24. Receiver 25 can be as simple as a bridge rectifier. Coil 26 can also be used as a transmitter to send status messages and confirmations. For added safety it may be desirable to build in a time delay in logic circuit 24 to delay arming by a set time (minutes to days)) after implantation in body. It was found that as long as wall 11 is below 50 um in thickness, it does not interfere significantly with signal reception by coil 26. The desired frequency range transmitted to coil 26 is 100 KHz-2 MHz. A small external coil, resonated by a capacitor, is used as a transmitter (not shown). The art of sending and receiving commands via inductive coupling or higher frequency RF signals is well known in the art and used in pacemakers. Clearly the arming and programming need not be done wirelessly. An insulated electrical contact on the outside of the defibrillator housing can be used to arm and program the device by touching an electrical wire to it.

In this disclosure the term “explosive” is used to describe any rapid chemical or physical reaction generating a mechanical shock. The explosive can be of the propellant type, such as smokeless gunpowder. Smokeless gunpowder is a common material made mainly of nitrocellulose and used in almost all firearms today. It can also be of the “high explosive” type, generating a high speed shock wave, such as nitroglycerine. Sometimes both types can be mixed for optimizing properties. It can be simply a very fast exothermic reaction generating a large volume of gas or steam. One especially desirable explosive is filling the sealed defibrillator with a compressed stoichiometric mixture of hydrogen and oxygen. Such a mixture, when ignited by igniter wire 17, creates a steam bubble which immediately disappears after cooling down. This may eliminate the need for flexible cover 10 in FIG. 2, as the condensed water can mix with the blood immediately. When using such an explosive mixture of compresses gasses, the thin wall 11 needs to be shaped as a dome or as a cylinder in order to resist the large storage pressure. The pressure is typically in the range of 3-50 Kg/cm². During combustions the pressure is many times higher, bursting the thin wall 11. It is desirable to make the housing 8 and wall 11 from a malleable corrosion resistant material such as type 316L stainless steel or titanium, in order to minimize the chance of any part separating from the housing during the explosion. It was found that the opening in wall 11 caused by the explosion is quite small, about 2-3 mm. In some cases the explosion can simply be caused by the release of a pressurized gas, without the need of a chemical reaction.

By the way of example, a miniature defibrillator according to the invention was tested in a simulated heart with the following results. The battery was two 1.55 V 5.8 mm diameter Silver Oxide watch batteries in series (the final device could use a custom made LI-Ion single cell 3V battery). The capacitor was a 500 uF/5V surface mount tantalum capacitor. The logic was constructed from CMOS logic to minimize power drain. The igniter wire was a 1 mm diameter coil having 2 turns of 0.05 mm diameter Nichrome wire. The explosive was standard smokeless gunpowder (removed from a 22 caliber cartridge). The flexible balloon was made of a regular latex toy balloon. Overall dimensions were about 6.5 mm diameter by 20 mm long. The housing was made from 0.15 mm type 316L stainless steel, ground down to about 0.05 mm thickness at the top part (to form the flexible wall) and resistance welded to form an hermetic seal. It was found that that about 50 mg of smokeless powder generated a thump judged sufficient for defibrillation. It was tested with 50 mg, 100 mg and 150 mg explosive charges. No damage to the surrounding simulated heart was detected. The amount of gas created was from about 2 cc to 6 cc. Some of the gas may have dissolved immediately into the saline solution simulating the blood. The same device was also tested with a stoichiometric mixture of hydrogen and oxygen compressed to 10 Kg/cm². The filling was done via a capillary tube that was pinched off after filling.

While the disclosure describes a miniature implanted device, an explosive charge generating a mechanical shock to the chest can be used as an external defibrillator. In such a device the explosive will be triggered mechanically by the user, in a manner used by all firearms. A spring loaded pin is released and strikes a primer which ignites the propellant. The generated gas is contained in a bag, similar to an automotive air bag. When unit is placed on the chest and a striking pin is released, the vigorous thump created by the rapidly inflated bag defibrillates the heart without damaging the body tissues. The main advantages of such a unit over present day external defibrillators are: very low cost, small size and unlimited shelf life. FIG. 6 shows the main components of an external unit placed over the chest above heart 1. An explosive cap 30, normally comprising of a primer and a propellant, is connected to a sealed flexible bag 31. A striking pin 33 in held in place by a safety pin 34. When pin 34 is pulled out, spring 32 causes pin 33 to detonate cap 30 and inflate bag 31, giving a thump to the heart. Since the bag can be folded, the complete unit can be stored in a minimal space and can be made disposable. An alternate propellant for inflating the bag is simply a container of pressurized gas. In this case pulling safety pin 34 ruptures the seal of the container and allows the gas to rapidly inflate bag 31. For external defibrillators worn by the user, automatic detection of erratic heartbeats can be added along the principles outlined for the implanted defibrillators. The defibrillator is automatically activated upon detecting such erratic heart rhythm, similar to an implanted device.

What is claimed is:

1. A cardiac defibrillator using a mechanical thump to defibrillate the heart when detecting an erratic heart rhythm.

2. A cardiac defibrillator capable of percutaneous delivery.

3. An explosive powered mechanical defibrillator.

4. A defibrillator as in claim 1 wherein said mechanical thump is created by an explosion.

5. A defibrillator as in claim 2 wherein said defibrillator comprises of a heartbeat sensor and an explosive charge.

6. A defibrillator as in claim 2 wherein the defibrillation is performed by a mechanical thump.

7. A defibrillator as in claim 2 wherein the defibrillation is performed by a mechanical thump created by an explosion.

8. A defibrillator as in claim 2 wherein the defibrillation is performed by a mechanical thump created by igniting a mixture of hydrogen and oxygen.

9. A defibrillator as in claim 3 wherein the explosive is a compressed mixture of hydrogen and oxygen

10. A defibrillator as in claim 2 wherein the defibrillator is housed in a hermetically sealed case covered by an inflatable flexible coating.

11. A defibrillator as in claim 1 capable of being armed by an external wireless signal.

12. A defibrillator as in claim 1 wherein said defibrillator is a disposable external device.

13. A defibrillator as in claim 3 wherein said defibrillator is a disposable external device.

14. A defibrillator as in claim 3 wherein said defibrillator is a disposable external device capable of automatic activation.

15. A defibrillator as in claim 3 wherein said defibrillator is a disposable external device using compressed gas as an explosive.

16. A defibrillator as in claim 2 capable of being delivered via a catheter of an internal diameter of 8 mm.

17. A defibrillator as in claim 2 having a sealed metal enclosure, said enclosure at least partially covered by an elastomeric balloon.

18. A defibrillator as in claim 1 having a sealed metal enclosure, said enclosure at least partially covered by an elastomeric balloon.

19. A defibrillator as in claim 3 activated by mechanical means.
